(19) United States
(12) Patent Application Publication
(10) Pub. No.: US 2007/0274579 A1
(43) Pub. Date: Nov. 29, 2007

(54) System And Method For Optimization Of Vessel Centerlines

(75) Inventors: Wenli Cai, Dorchester, MA (US); Frank C. Dachille, Wailea, HI (US)

Correspondence Address: Frank Chau;F CHAU & ASSOCIATES, 130 Woodbury Road, Woodbury, NY, 11797, US

(73) Assignee: VIATRONIX INCORPORATED, Stony Brook, NY (US)

(21) Appl. No.: 10/580,772

(22) PCT Filed: Nov. 24, 2004
(86) PCT No.: PCT/US04/39895

§ 371 Date: Apr. 16, 2007

Related U.S. Application Data
(60) Provisional application No. 60/525,603, filed on Nov. 26, 2003.

(51) Int. Cl. A61B 6/00

(52) U.S. Cl. 382/131

(57) ABSTRACT

Methods are provided for optimizing a vessel centerline in a digital image. For instance, a method includes providing a digital image of a vessel wherein said image comprises a plurality of intensities corresponding to a domain of points in a D-dimensional space, initializing a centerline comprising a plurality of points in the vessel (step 20), determining a cross section of the vessel at each point in the centerline (step 21), evaluating a center point for each cross section of the vessel (step 22), and determining a refined centerline from the center points of each cross section (step 23).

### CROSS REFERENCE TO RELATED APPLICATION

This application claims priority to U.S. Provisional Application Ser. No. 60/525,603 filed Nov. 26, 2003, the contents of which are fully incorporated herein by reference.

### TECHNICAL FIELD

This invention is directed to the analysis of digital images, particularly digital medical images.

### DISCUSSION OF THE RELATED ART

Analysis of vascular structures acquired by computerized tomographic angiography (CTA) or magnetic resonance angiography (MRA) is commonly performed for clinical diagnosis of vascular disease, e.g. assessing and monitoring stenosis secondary to atherosclerosis, for surgery planning, etc. Vessels can be evaluated using computerized tomographic (CT) and magnetic resonance (MRI) imaging modalities quantitatively—for example, stenosis can be calculated by ratios of minimum to normalized diameter or cross-sectional area. Blood vessels can also be evaluated qualitatively using volume and surface rendering post-processing. Based on the tubular shape of vessels, a geometric model for vascular quantification utilizes a centerline and a series of cross-sections perpendicular to the centerline. Cross-sectional diameters and areas can then be calculated. An automatic reproducible vascular quantification relies on an automatic, reproducible and accurate centerline.

The process to extract vessel centerline and its associated cross-sections is called vessel skeletonization. Skeletonization simplifies the shape of a vessel to the closest set of centers of maximal inscribed disks, which can fit within the object. The central locus of the centers is made the centerline.

There exists a wide variety of 3D skeletonization algorithms based on different definitions and extraction approaches. In the context of vessel skeletonization, many centerline extraction methods have been developed. There are three basic approaches to centerline extraction based on input data: (1) binary data; (2) distance map; and (3) raw data. A good skeletonization preserves the topology of the original shape, and approximates the central axis. The resulting central axis should be thin, smooth and continuous, and allow full object recovery.

A vessel centerline extraction technique should be able to handle noisy data, branches, and complex blood vessel anatomy. Generally speaking, centerline algorithms detect bright objects on dark background. But due to calcification, there are some high intensity spots (known as plaques) within vessels in CTA data sets, particularly in elderly patients due to advanced atherosclerosis. Plaques are located within vessel walls and thus change the profile of local signal intensities. They can be mistaken as part of the vessel lumen (missing the real lumen) or as part of bones (missing the plaques). A centerline should be centered based on the vessel walls and should also not break or twist due to obstructions caused by plaques and/or high-grade stenoses. Most of the current centerline algorithms have difficulties overcoming plaques in CTA studies.

Another reason that the normal, discrete one-voxel-wide (some half-voxel-wide) centerline is not satisfactory in clinical applications is the non-reproducibility of vessel quantification. Quantification relies on an accurate and reproducible centerline. In fact, when one vessel is measured by different users or measured at different times or measured by different algorithms, the centerline may vary. This non-reproducibility or inaccuracy of quantification weakens its clinical application. Hence, in order to attain reproducible quantification, centerlines need to be optimized to approximate the central axes, i.e., a good skeletonization. Most current algorithms use smoothing after centerline extraction in order to remove the jagged changes in the centerline. But smoothing does not maintain centralization of the vessel skeleton in extracting the true centerlines in CTA studies. In some cases non-perpendicular cross-sections result in a twisted or crooked centerline by changing the connecting order of center points. This correlation between orientation and center of a cross-section is one of the main drawbacks in vessel tracking. The centerline also needs to be refined after being extracted. Refinement is an optimization process to approximate the centerline to the central axis, called the optimal centerline and also known as the good skeletonization.

### SUMMARY OF THE INVENTION

Exemplary embodiments of the invention as described herein generally include methods and systems for extracting and refining centerlines using a distance map, referred to herein as the distance to boundary (DTB) volume, where the centerline is defined to be the center of vessel's walls, including lumen and plaque, rather than only its lumen.

In accordance with the invention, there is provided a method of optimizing a vessel centerline in a digital image including the steps of providing a digital image of a vessel wherein said image comprises a plurality of intensities corresponding to a domain of points in a D-dimensional space, initializing a centerline comprising a plurality of points in the vessel, determining a cross section of the vessel at each point in the centerline, evaluating a center point for each cross section of the vessel, and determining a refined centerline from the center points of each cross section.

In a further aspect of the invention, the steps of determining a cross section, evaluating a center point, and determining the refined centerline are repeated until the difference between each pair of successive refined centerlines is less than a predetermined quantity.

In a further aspect of the invention, the cross section at a point in the centerline is determined by finding a cross section intersecting the centerline with a minimal area.

In a further aspect of the invention, the cross section with minimal area is the cross section with the shortest lines intersecting the point in the centerline.

In a further aspect of the invention, the cross section at a point on the centerline is perpendicular to a tangent vector of the centerline at the point on the centerline.

In a further aspect of the invention, the method further comprises associating a reference frame to each cross section, wherein each said reference frame is defined by the centerline point in the cross section, and three orthogonal vectors that define an orientation of the reference frame, wherein the three orthogonal vectors include a tangent to the centerline at the centerline point, and two other orthogonal vectors in the plane of the cross section.

In a further aspect of the invention, a first referenced frame can be determined from the centerline point in the cross section and the three orthogonal vectors, and a next reference frame can be determined by displacing the first reference frame to a next centerline point and rotating the displaced reference frame to align with the three orthogonal vectors of the cross section associated with the next centerline point.

In a further aspect of the invention, evaluating a center point of each cross section comprises finding the contour of the cross section and using the contour to locate the centerpoint of the cross section.

In a further aspect of the invention, evaluating a center point of each cross section comprises calculating a centroid of each cross section.

In a further aspect of the invention, the method further comprises calculating the covariance matrix for each cross section, and calculating the eigenvalues and eigenvectors of the covariance matrix to determine the shape of the cross section.

In a further aspect of the invention, determining a refined centerline further includes connecting each successive pair of center points by a virtual spring whose force depends on the difference of the orientations of the pair of center points, applying a stochastic perturbation to each virtual spring, determining an optimized cross section of minimal area for each point on the centerline, finding a center point of the optimized cross section, and forming a refined centerline by connecting the center points of each optimized cross section.

In a further aspect of the invention, the refined centerline is approximated by a least square cubic curve.

In a further aspect of the invention, finding a center point of the optimized cross section comprises calculating a centroid of each optimized cross section.

In a further aspect of the invention, the spring force connecting two successive centerpoint is defined by ƒ=k (1.0−T₀•T₁), wherein k is a constant and T₀ and T₁ are the tangent vectors of two successive center points.

In a further aspect of the invention, the method further comprises the step of refining the centerline until it has converged to an optimal centerline, wherein convergence is determined from the displacement of each center point and the deviation of the orientation of each reference plane.

In a further aspect of the invention, convergence is determined by considering a maximum of the displacement and orientation as defined by
(DSₘₐₓ^{k}DVₘₐₓ^{k})=maxᵢ₌₁ⁿ(|Cᵢ^{k}−Pᵢ^{k}|,1−Tᵢ^{k}•Nᵢ^{k}),
where DSₘₐₓ^{k} is the maximum displacement and DVₘₐₓ^{k} is the maximum deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

In a further aspect of the invention, convergence is determined by considering an average of the displacement and orientation as defined by

$$( {DS}_{avg}^{k} , {DV}_{avg}^{k} ) = \frac{1}{N} {\sum }_{i = 1}^{n} ( \vert {C}_{i}^{k} - {P}_{i}^{k} \vert , 1 - {T}_{i}^{k} \cdot {N}_{i}^{k} )$$

where DS_{avg}^{k} is the average displacement and DV_{avg}^{k} is the average deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

In a further aspect of the invention, the method further includes calculating the lumen and wall contours on each cross-section, as well as other geometric information about these two contours.

In a further aspect of the invention, the method further comprises the step of providing an endoluminal flight along the centerline of a vessel object, displaying hard plaque and soft plaque in different colors for differentiation from the vessel wall.

In a further aspect of the invention, the method further comprises moving back and forth along the centerline by direct manipulation of a mechanism.

In a further aspect of the invention, the mechanism includes clicking or dragging a mouse along an overview of the entire vessel or scrolling a mouse wheel to scroll along the centerline of the vessel.

In a further aspect of the invention, the mechanism includes interactively tilting a viewpoint without leaving the centerline of the vessel.

In another aspect of the invention, there is provided a program storage device readable by a computer, tangibly embodying a program of instructions executable by the computer to perform the method steps for optimizing a vessel centerline in a digital image.

These and other exemplary embodiments, features, aspects, and advantages of the present invention will be described and become more apparent from the detailed description of exemplary embodiments when read in conjunction with accompanying drawings.

### BRIEF DESCRIPTION OF THE DRAWINGS

FIG. 1 is an exemplary diagram illustrating a method for defining a generalized centerline according to an exemplary embodiment of the invention.

FIG. 2 depicts a flow diagram illustrating a centerline refinement process, according to an exemplary embodiment of the invention.

FIG. 3 is an exemplary diagram illustrating a method for computing a cross sectional line given a center point of a circle.

FIG. 4 is an exemplary diagram that illustrating a method for computing a minimum cross-sectional area perpendicular to the central axis of a cylinder.

FIG. 5 depicts a method for centerline convergence according to an exemplary embodiment of the invention.

FIG. 6 depicts a method for computing reference frames of successive center points along a centerline, according to an exemplary embodiment of the invention.

FIG. 7 depicts a method for determining the cross-section of a distance-to-boundary field, according to an exemplary embodiment of the invention.

FIG. 8 depicts a method for determining the centroid of the cross-section, according to an exemplary embodiment of the invention.

FIG. 9 depicts a method for coupling local cylinders, according to an exemplary embodiment of the invention.

FIG. 10 depicts a flow diagram illustrating a centerline refinement process, according to another exemplary embodiment of the invention.

### DETAILED DESCRIPTION OF THE EXEMPLARY EMBODIMENTS

Exemplary embodiments of the invention are described below. In the interest of clarity, not all features of an actual implementation which are well known to those of skill in the art are described in detail herein.

It is to be understood that the present invention may be implemented in various forms of hardware, software, firmware, special purpose processors, or a combination thereof. Preferably, the present invention is implemented as a combination of both hardware and software, the software being an application program tangibly embodied on a program storage device. The application program may be uploaded to, and executed by, a machine comprising any suitable architecture. Preferably, the machine is implemented on a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device.

It is to be further understood that, because some of the constituent system components depicted in the accompanying Figures may be implemented in software, the actual connections between the system components may differ depending upon the manner in which the present invention is programmed. Given the teachings herein, one of ordinary skill in the related art will be able to contemplate these and similar implementations or configurations of the present invention.

### Overview of the Methods

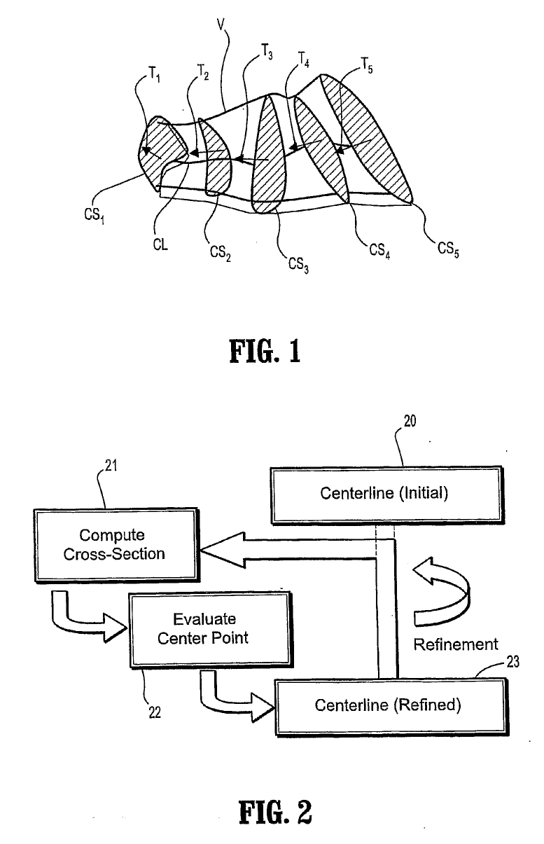

FIG. 1 is an exemplary diagram illustrating a method for defining a generalized centerline according to an exemplary embodiment of the invention. In order to define a centerline, a vessel can be represented by a narrow tubular structure, which in general is a cylinder, as depicted in the figure. Then, the centerline can be regarded as the central curve axis of the cylinder. At each point of the central axis there is a cross-section that is perpendicular to the axis, i.e., the center of the cross-section is on the centerline; the normal of the cross-section is the tangent of the centerline at this point. Hence, the centerline can be defined to consist of the centers of the cross-sections. FIG. 1 depicts centerline CL of vessel V, connecting cross sections CS₁, CS₂, CS3, CS₄, and CS₅, with normals T₁, T₂, T₃, T₄, and T₅, respectively, that are tangent to the centerline where the centerline CL intersects each cross section.

However, this definition of centerline is recursive: (1) A centerline is a closure set of centers of the cross-sections of the object; and (2) A cross-section is a cut plane that is perpendicular to the centerline. A cross-section is needed to compute a center point, but the position and orientation of a cross-section is defined by a segment of centerline, which is approximated or interpolated by a set of center points.

FIG. 2 depicts a flow diagram illustrating a centerline refinement process, according to an exemplary embodiment of the invention. In general, a refinement process approximates the central axis by iteratively adjusting the points towards the cross-section centers, i.e. the optimal centerline. Referring to the figure, an initial step 20 is to compute an initial centerline (which may be inaccurate). Then, a next step 21 is to compute the cross-sections of the initial centerline, followed by evaluating the center on each cross-section at step 22, then updating the centerline by the center points evaluated at step 23. Returning to step 21, the new cross-sections will be computed according to the updated centerline. This refinement process can continue until the changes between successive loops is less than a desired accuracy, i.e. when it converges to the optimal centerline.

To compute a cross section given a center point, consider a vessel segment that is a cylinder. In this case, the cross-section at a center point is defined by the position (P) and the orientation (or tangent vector) (α) at this point. Thus, the area (S) of cross-sections within this segment is a function of P and α, i.e. S(P, α). The cross-section that is perpendicular to the centerline has the minimum area, i.e. min_{α}{S(P, α)}. The tangent vector of a centerline at a center point is always perpendicular to the cross-section through the center point that has the minimal cross-sectional area. The local minimum area ensures a unique convergent position. Therefore, the centerline refinement is an optimization process to find the orientation of minimum cross-sectional area within each segment, i.e. a cylinder with the centerline having n segments, where Sᵢ is the cross-sectional area at segment i.

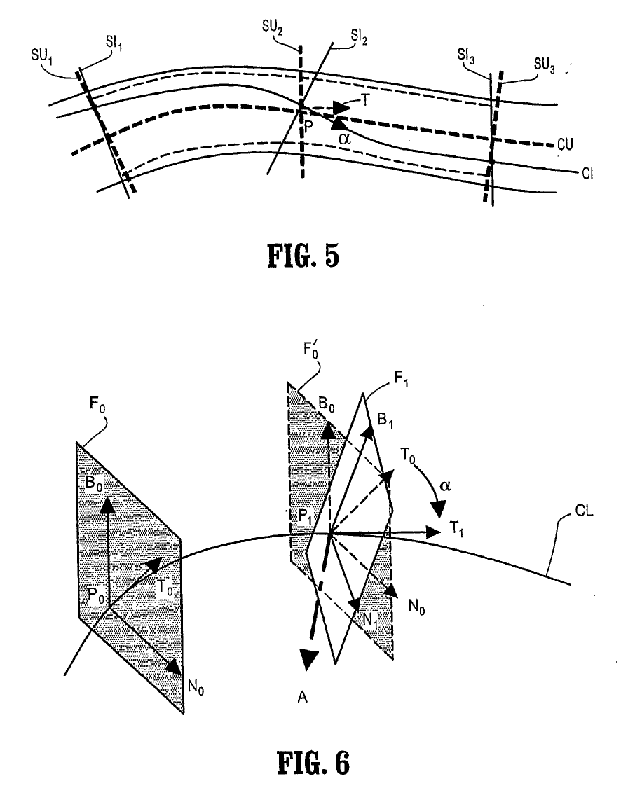

FIG. 5 depicts a method for centerline convergence according to an exemplary embodiment of the invention. An initial centerline CI has initial cross sections SI₁, SI₂, and SI₃. At each center point a local general cylinder, whose boundary is indicated by a in the figure, is set up with ellipse parameters extracted from the neighboring center points. The local general cylinder can be used to update the refined cross sections SU₁, SU₂, and SU₃, which determine the refined centerline CU. By way of example, updated centerline CU has center point P in updated cross section SU₂. The vector T is tangent to the updated center line CU at point P and is perpendicular to the updated cross section SU₂.

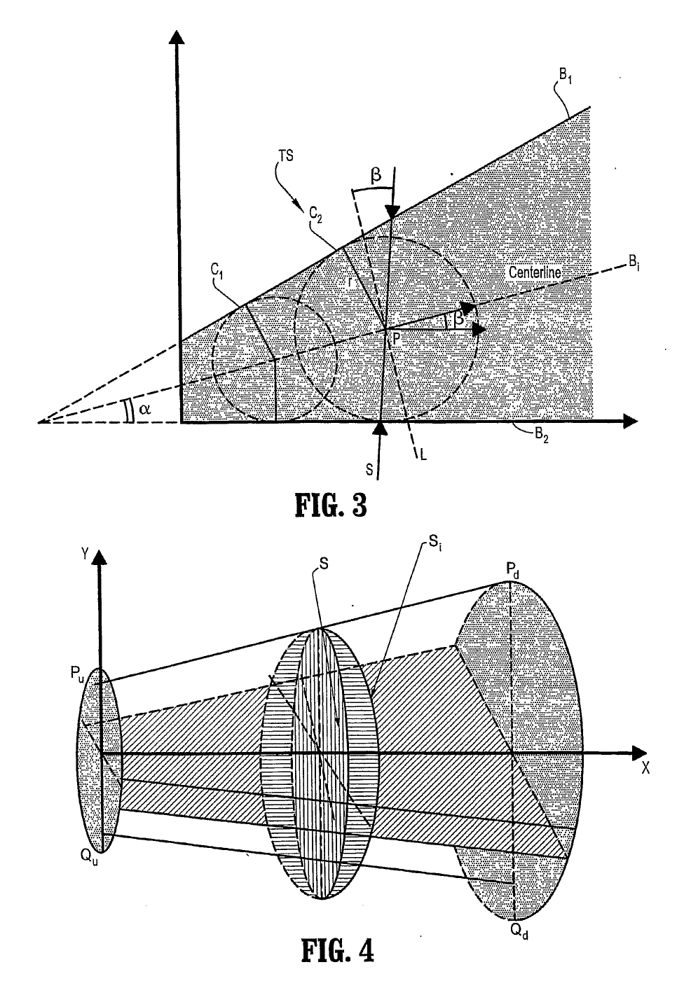

In order to see why the appropriate cross section is the cross section with minimal cross sectional area, consider a 2D case. FIG. 3 is an exemplary diagram illustrating a method for computing a cross sectional line of a circle given a center point. The figure depicts a tubular structure TS whose boundaries vary linearly within a small range, as indicated by two circles, C₁ and C₂. One boundary B₁ can be located on the x-axis and another boundary B₂ on another line as shown in the figure. If these two boundaries are parallel, then the minimum length cross-sectional-line is perpendicular to the centerline, which is located at the middle of these two boundaries and is parallel to the boundaries. However, as depicted in the figure, if the two boundaries are not parallel, the centerline is actually the angular bisector of the angle formed by the two boundaries B₁, B₂. Now, suppose that P is a point on the angular bisector Bᵢ: the angle between an arbitrary oblique cross-sectional-line S and the perpendicular cross-sectional-line L is β; the distance from P to the boundaries is r; thus, the length of the oblique cross-sectional-line is

$$S = \frac{r}{cos ( β + α )} + \frac{r}{cos ( β - a )} .$$

The shortest intersection line is found when β=0.

An analogous result can be obtained in the 3D case. FIG. 4 is an exemplary diagram that illustrating a method for computing a minimum cross-sectional area perpendicular to the central axis of a cylinder. When quadrilateral PᵤQᵤQ_{d}P_{d} is rotated around the X axis, cross-section S that is perpendicular to central axis (X axis) always contains the shortest intersection line compared to other cross-sections Sᵢ that are not perpendicular to the central axis. The area of the cross-section is the integral of the area of all fans along the contours. Thus, the shortest intersection lines results in the minimum cross-sectional area.

This concept of minimal cross-sectional area is reasonable in clinical practice. There are many possible orientations and positions of an oblique cut plane within a small segment of a vessel. In terms of stenosis detection, the plane of most interest is the one with minimum cross-sectional area.

### Initializing the Centerline

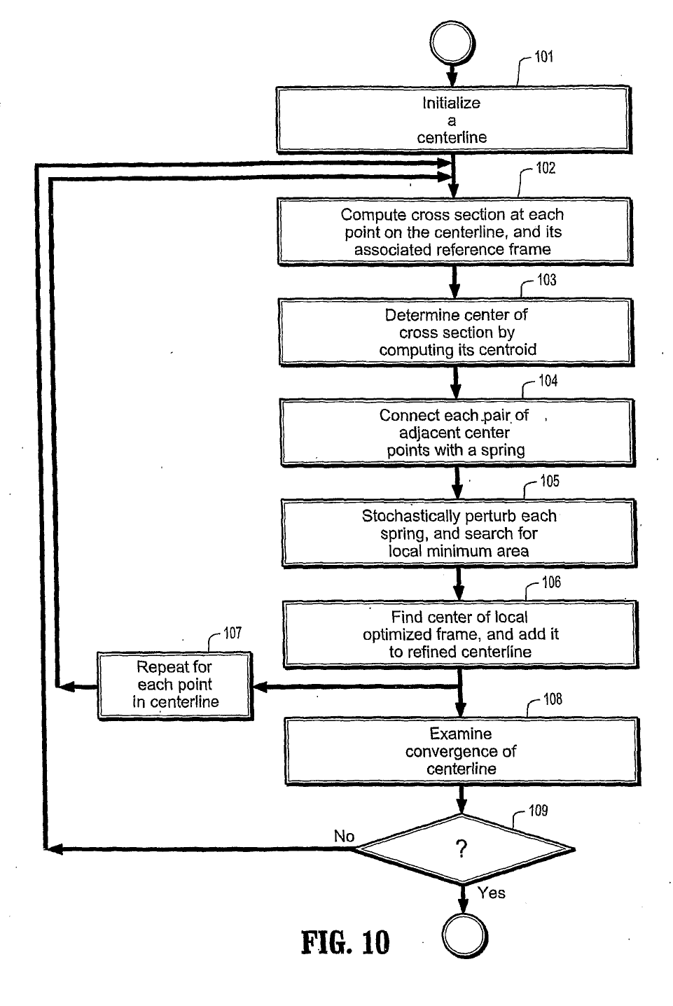

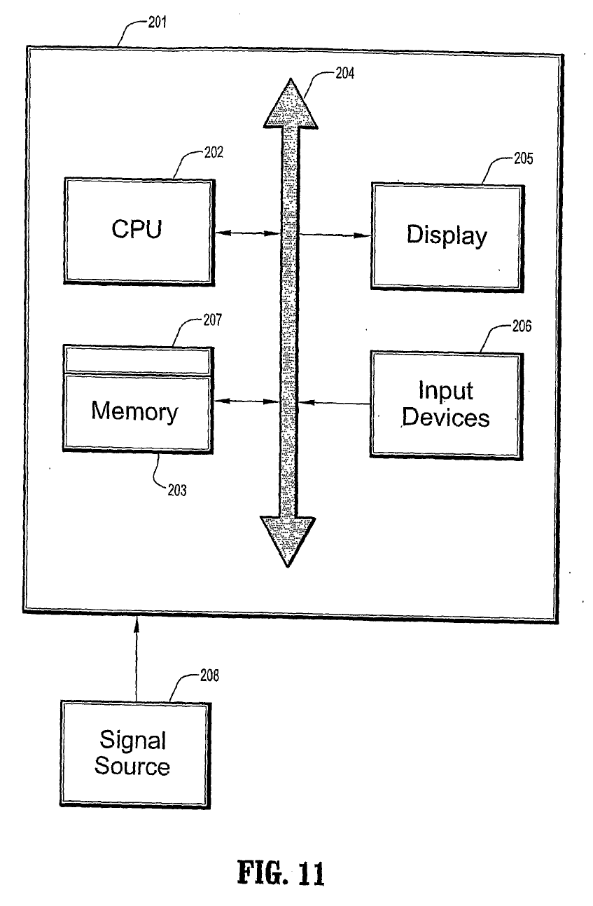

FIG. 10 depicts a flow diagram illustrating a centerline refinement process, according to an exemplary embodiment of the invention depicted in FIG. 2. Referring now to the flowchart depicted in FIG. 10, a centerline can be initialized at step 101 using any centerline initialization algorithm known in that art or even via hand-drawing a piecewise linear centerline. Different centerline algorithms do not significantly affect the results of a refinement process according to the invention, but might affect the computation time. The initial centerline need not be accurate but should be located within the object. In one embodiment of the procedure, a method such as that disclosed in U.S. Patent Application Publication 2004/0109603, which is well known in the art, is used to create the initial centerline.

The centerline is divided into a number of line segments, for each of which a minimum cross-sectional area is evaluated. This division is done via parameterization of the initial centerline. The initial discrete centerline is first approximated by a cubic spline. In one embodiment of the invention, the splines are NURBS curves. Then, the approximated curve is re-sampled equidistantly with a pre-defined arc-length λ to create a new discrete set of center points. In one embodiment of the invention, the arc length is 2 mm. Each re-sampled center point represents a small centerline segment of length λ. The tangent vector of the centerline is the initial orientation of the cross-section at that point.

A next step 102 is to compute a cross section at each point on the centerline, and an associated reference frame. Assuming that the vessels are not severely twisted, a vessel can be constructed by extruding a reference frame among cross-sections along the centerline.

FIG. 6 depicts a method for computing reference frames of successive center points along a centerline CL, according to an exemplary embodiment of the invention. A reference frame F₀ comprises a reference point P₀, the position of the frame on the centerline, and a set of three orthogonal axes (T₀, B₀, N₀) that define the orientation, as illustrated in FIG. 6. T is the unit tangent vector of the centerline; B is the bi-normal vector and N is the principal normal vector. The initial reference frame F₀ can be computed based on the curvature of the centerline. Given the initial frame F₀, a subsequent frame F₁ specified by {(P₁, (T₁, B₁, N₁)} can be computed by minimizing the torsion among its neighbors, as shown in the figure. First, a rotation axis A is selected and a rotation matrix is computed using T₀ and T₁. Then the initial frame (P₀, T₀) is rotated through an angle a such that the T₀ aligns itself with the T₁. This rotation creates a new N and B. By moving the rotated frame to P₁, a new frame (P₁, T₁) is created with the minimum torsion to P₀. By way of comparison, FIG. 6 also depicts the frame F₁′ formed by simply displacing initial frame F₀ is displaced to position P₁ without rotation, superimposed on new frame F₁. Because vessels are asymmetric, especially at the location of plaques, cross-section alignment with minimized torsion is helpful to ensure a correct local generalized cylinder.

Each reference frame F₀, F₁, corresponds to a cross-section of a centerline. A generalized cylinder can be constructed from the cross-sections, which are properly centered on the central axis.

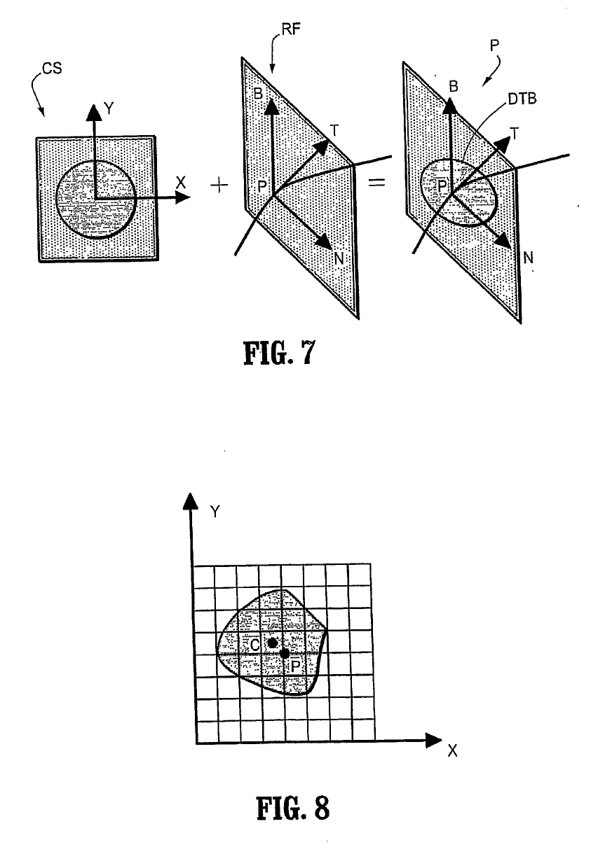

FIG. 7 depicts a method for relating the cross-section to an oblique cut plane in space, according to an exemplary embodiment of the invention. The x- and y-axis of a cross-section CS can be aligned with, respectively, the N and B vector of reference frame RPF to form an oblique cut plane P in space. This plane P is filled in to the distance-to-boundary (DTB) volume, as illustrated in FIG. 7.

Referring again to FIG. 10, a next step 103 is to determine the center of a cross section by computing its centroid. The center of a cross-section of a generalized cylinder is the center point of the central curve axis, i.e. the optimal centerline. In general, the center of a cross-section can be the geometric center or the physical centroid. One method to compute the center point is to find all of the boundary pixels in the cross-section, i.e. the contour, and calculate the center point by using the detected contour. Another method used in an exemplary embodiment of the invention uses a central moment to estimate the center of a DTB cross-section.

FIG. 8 depicts a method for determining the centroid of the cross-section, according to an exemplary embodiment of the invention. Suppose that a DTB cross-section is a 2D discrete function ƒ(x, y). Then, the ijth moment about zero is defined as:

$${m}_{ij} = \frac{{\sum }_{x = 1}^{N} {\sum }_{y = 1}^{N} {x}^{i} {y}^{j} f ( x , y )}{{\sum }_{x = 1}^{N} {\sum }_{y = 1}^{N} f ( x , y )} .$$

The x and y components (μₓ, μ_{y}) of the mean can be defined by
(μₓ, μ_{y})=(m₀₁, m₁₀),
so that (μₓ, μ_{y}) is the centroid point C, where point P is the center of the reference frame. As shown in FIG. 8, the centroid point C does not necessarily coincide with the reference point P. Thus the initial point can be located outside the vessel contour as long as the cross-section contains the vessel to be refined.

Furthermore, the central moments μᵢⱼ can be defined as below:

$${μ}_{ij} = \frac{{\sum }_{x = 1}^{N} {\sum }_{y = 1}^{N} {( x - {μ}_{x} )}^{i} {( y - {μ}_{y} )}^{j} f ( x , y )}{{\sum }_{x = 1}^{N} {\sum }_{y = 1}^{N} f ( x , y )}$$

The covariance matrix is

$$[ \begin{matrix}{μ}_{20} & {μ}_{11} \\ {μ}_{11} & {μ}_{02}\end{matrix} ]$$

where moments μ₂₀ and μ₀₂ are the variance of x and y, μ₁₁ is the covariance between x and y. By finding the eigenvalues and eigenvectors of the covariance matrix, one can estimate the shape of a cross-section, including the short axis, the long axis, the eccentricity, the elongation, and the orientation of the shape, assuming it is in general an ellipse. Using these shape parameters, a local cylinder can be constructed on the current cross-section and its neighbors.

Referring once again to FIG. 2, the next step is to refine the centerline, as indicated by step 23. In an ideal situation, the position and the tangent vector of a local central curve axis could be directly calculated if all the cross-sections are symmetric. More generally, the central axis can be approximated via a local minimal cross-sectional area. In one embodiment of the invention, the optimization model is a spring model with a stochastic perturbation. Referring back to FIG. 10, the steps 104, 105, and 106 form one exemplary embodiment of step 23 of the embodiment illustrated in FIG. 2.

Referring to FIG. 10, the next step 104 is to connect each pair of adjacent center points with a spring. However, instead of considering the displacement between two points, since each point is limited within its local cylinder due to the equidistant re-parameterization, the spring force is a function of the difference between two orientations: ƒ=k (1.0−T₀•T₁).

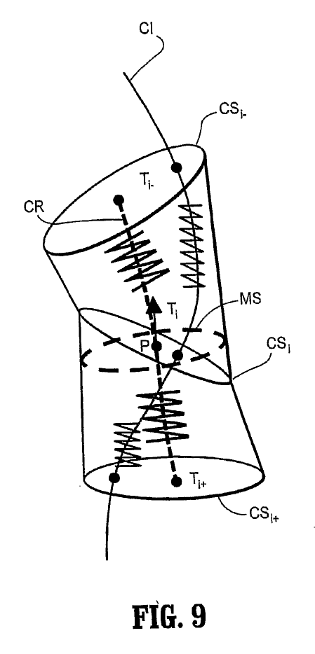

FIG. 9 depicts a method for coupling local cylinders, according to an exemplary embodiment of the invention. Referring now to FIG. 9, a cross-section CSᵢ on the input centerline CI is coupled by spring forces to both cross-section CSᵢ₊ and CSᵢ₋. The stable orientation is defined by a weighted summation of Tᵢ, Tᵢ₊ and Tᵢ₋, where the weight is the spring coefficient.

In each iteration step, the cross-sectional orientations are adjusted by the spring forces. Step 105 stochastically perturbs each spring, and searches for a local minimum area. A minimal area cross section MS for cross section i is indicated by dashed circle in FIG. 9. Step 106 finds the center of the local optimized frame, and adds it to the refined centerline. The center of the local optimized frame is taken as the refined center point, a refined centerline CR is formed from the local central curve axis, as indicated in FIG. 9. Accordingly, the centerline is refined with the goal of minimum cross-sectional area constrained to the spring forces. The new centerline is approximated globally and re-sampled to a set of center points after one loop. In one exemplary embodiment of the invention, the global approximation is by a least square cubic curve.

At step 107 the preceding steps are repeated for each point on the centerline. The steps depicted in FIG. 10 are exemplary, and variations that will be apparent to those skilled in the art are within the scope of the invention. For example, each of the steps 102, 103, 104, 105, and 106 could be performed for each point in the centerline before moving on to the next step.

### Convergence of the Refinement

A next step 108 is to examine convergence of centerline. The criteria of convergence are the displacement of the center point and the deviation of the orientation (normal vector) of the reference frame. Although the minimum cross-sectional area is used to optimize the local center point, the sum of all cross-sectional areas cannot be taken as the global property of the optimum due to the following facts. First, the reference frame is equidistantly positioned on the centerline. During optimization, center points are adjusted and the curve length of the centerline varies. Thus the number and the position of the reference frames may vary at each iteration step. Second, since the position of the frame varies at each iteration step and the local cross-sectional area of the object is inconsistent, the local minimum cross-sectional area has no consistency among different iterations.

For these reasons, both the displacement of the center points and the deviation of the tangent vector of a centerline are taken as the factors of convergence. If both are less than a pre-defined threshold after the iteration steps, the centerline can be considered convergent. Both the maximum and average of the displacement and deviation are considered. These convergence factors can be expressed as

$$( {DS}_{max}^{k} , {DV}_{max}^{k} ) = {max}_{i = 1}^{n} ( \vert {C}_{i }^{k} - {P}_{i}^{k} \vert , 1 - {T}_{i}^{k} \cdot {N}_{i}^{k} ) , \\ ( {DS}_{avg}^{k} , {DV}_{avg}^{k} ) = \frac{1}{N} {\sum }_{i = 1}^{n} ( \vert {C}_{i }^{k} - {P}_{i}^{k} \vert , 1 - {T}_{i}^{k} \cdot {N}_{i}^{k} )$$

where, for the k^{th} iteration, DS is the i^{th} displacement and DV is the deviation of the i^{th} tangent vector, C is the i^{th} updated center point, P is the position of the i^{th} reference frame, T is the i^{th} updated tangent direction and N is the normal of the i^{th} reference frame.

If, at step 2209, it is determined that that centerline has not converged, the refinement process is repeated.

The methods discloses herein have evaluated using both phantom data sets and clinical data sets. Phantom data sets are used to evaluate the expected properties of the methods as well as their accuracy. The clinical data sets are used to evaluate the methods in practice, mainly for their reproducibility. These tests have demonstrated the effectiveness, reproducibility and stability of the methods herein disclosed for determining a vessel centerline.

### System Implementations

It is to be understood that the present invention can be implemented in various forms of hardware, software, firmware, special purpose processes, or a combination thereof. In one embodiment, the present invention can be implemented in software as an application program tangible embodied on a computer readable program storage device. The application program can be uploaded to, and executed by, a machine comprising any suitable architecture.

It is to be understood that the methods described above may be implemented using various forms of hardware, software, firmware, special purpose processors, or a combination thereof. Preferably, the present invention is implemented as a combination of both hardware and software, the software being an application program tangibly embodied on a program storage device. The application program may be uploaded to, and executed by, a machine comprising any suitable architecture. Preferably, the machine is implemented on a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device.

It is to be further understood that since the exemplary systems and methods described herein can be implemented in software, the actual method steps may differ depending upon the manner in which the present invention is programmed. Given the teachings herein, one of ordinary skill in the related art will be able to contemplate these and similar implementations or configurations of the present invention.

Indeed, while the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A method of optimizing a vessel centerline in a digital image, said method comprising the steps of:
providing a digital image of a vessel wherein said image comprises a plurality of intensities corresponding to a domain of points in a D -dimensional space;
initializing a centerline comprising a plurality of points in the vessel;
determining a cross section of the vessel at each point in the centerline;
evaluating a center point for each cross section of the vessel; and
determining a refined centerline from the center points of each cross section.

2. The method of claim 1, wherein the steps of determining a cross section, evaluating a center point, and determining the refined centerline are repeated until the difference between each pair of successive refined centerlines is less than a predetermined quantity.

3. The method of claim 1, wherein the cross section at a point in the centerline is determined by finding a cross section intersecting the centerline with a minimal area.

4. The method of claim 3, wherein the cross section with minimal area is the cross section with the shortest lines intersecting the point in the centerline.

5. The method of claim 1, wherein the cross section at a point on the centerline is 25 perpendicular to a tangent vector of the centerline at the point on the centerline.

6. The method of claim 5, further comprising associating a reference frame to each cross section, wherein each said reference frame is defined by the centerline point in the cross section, and three orthogonal vectors that define an orientation of the reference frame, wherein the three orthogonal vectors include a tangent to the centerline at the centerline point, and two other orthogonal vectors in the plane of the cross section.

7. The method of claim 6, wherein a first referenced frame can be determined from the centerline point in the cross section and the three orthogonal vectors, and a next reference frame can be determined by displacing the first reference frame to a next centerline point and rotating the displaced reference frame to align with the three orthogonal vectors of the cross section associated with the next centerline point.

8. The method of claim 1, wherein evaluating a center point of each cross section comprises finding the contour of the cross section and using the contour to locate the centerpoint of the cross section.

9. The method of claim 1, wherein evaluating a center point of each cross section comprises calculating a centroid of each cross section.

10. The method of claim 9, further comprising calculating the covariance matrix for each cross section, and calculating the eigenvalues and eigenvectors of the covariance matrix to determine the shape of the cross section.

11. The method of claim 6, wherein determining a refined centerline further comprises the steps of:
connecting each successive pair of center points by a virtual spring whose force depends on the difference of the orientations of the pair of center points,
applying a stochastic perturbation to each virtual spring;
determining an optimized cross section of minimal area for each point on the centerline;
finding a center point of the optimized cross section; and
forming a refined centerline by connecting the center points of each optimized cross section.

12. The method of claim 11, wherein the refined centerline is approximated by a least square cubic curve.

13. The method of claim 11, wherein finding a center point of the optimized cross section comprises calculating a centroid of each optimized cross section.

14. The method of claim 11, wherein the spring force connecting two successive centerpoint is defined by ƒ=k (1.0−T₀•T₁), wherein k is a constant and T₀ and T₁ are the tangent vectors of two successive center points.

15. The method of claim 11, further comprising the step of refining the centerline until it has converged to an optimal centerline, wherein convergence is determined from the displacement of each center point and the deviation of the orientation of each reference plane.

16. The method of claim 15, wherein convergence is determined by considering a maximum of the displacement and orientation as defined by
(DSₘₐₓ^{k}DVₘₐₓ^{k})=maxᵢ₌₁ⁿ(|Cᵢ^{k}−Pᵢ^{k}|,1−Tᵢ^{k}•Nᵢ^{k}),
where DSₘₐₓ^{k} is the maximum displacement and DVₘₐₓ^{k} is the maximum deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

17. The method of claim 15, wherein convergence is determined by considering an average of the displacement and orientation as defined by
$$( {DS}_{avg}^{k} , {DV}_{avg}^{k} ) = \frac{1}{N} {\sum }_{i = 1}^{n} ( \vert {C}_{i }^{k} - {P}_{i}^{k} \vert , 1 - {T}_{i}^{k} \cdot {N}_{i}^{k} )$$
where DS_{avg}^{k} is the average displacement and DV_{avg}^{k} is the average deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

18. The method of claim 5, further including calculating the lumen and wall contours on each cross-section, as well as other geometric information about these two contours.

19. The method of claim 1, further comprising the step of providing an endoluminal flight along the centerline of a vessel object, displaying hard plaque and soft plaque in different colors for differentiation from the vessel wall.

20. The method of claim 19, further comprising moving back and forth along the centerline by direct manipulation of a mechanism.

21. The method of claim 20, wherein the mechanism includes clicking or dragging a mouse along an overview of the entire vessel or scrolling a mouse wheel to scroll along the centerline of the vessel.

22. The method of claim 20, wherein the mechanism includes interactively tilting a viewpoint without leaving the centerline of the vessel.

23. A method of optimizing a vessel centerline in a digital image, said method comprising the steps of:
providing a digital image of a vessel wherein said image comprises a plurality of intensities corresponding to a domain of points in a D-dimensional space;
initializing a centerline comprising a plurality of points in the vessel;
determining a cross section of the vessel at each point in the centerline, wherein the cross section at a point on the centerline is perpendicular to a tangent vector of the centerline at the point on the centerline;
associating a reference frame to each cross section, wherein each said reference frame is defined by the centerline point in the cross section, and three orthogonal vectors that define an orientation of the reference frame, wherein the three orthogonal vectors include a tangent to the centerline at the centerline point, and two other orthogonal vectors in the plane of the cross section;
evaluating a center point for each cross section of the vessel by calculating a centroid of each cross section;
connecting each successive pair of center points by a virtual spring whose force is defined by ƒ=k (1.0−T₀•T₁), wherein k is a constant and T₀ and T₁ are the tangent vectors of two successive center points;
applying a stochastic perturbation to each virtual spring;
determining an optimized cross section of minimal area for each point on the centerline;
finding a center point of the optimized cross section by calculating its centroid;
forming a refined centerline by connecting the center points of each optimized cross section; and
refining the centerline until it has converged to an optimal centerline, wherein convergence is determined from the displacement of each center point and the deviation of the orientation of each reference plane.

24. The method of claim 23, wherein a first referenced frame can be determined from the centerline point in the cross section and the three orthogonal vectors, and a next reference frame can be determined by displacing the first reference frame to a next centerline point and rotating the displaced reference frame to align with the three orthogonal vectors of the cross section associated with the next centerline point.

25. The method of claim 23, further comprising calculating the covariance matrix for each cross section, and calculating the eigenvalues and eigenvectors of the covariance matrix to determine the shape of the cross section.

26. The method of claim 23, wherein the refined centerline is approximated by a least square cubic curve.

27. The method of claim 23, wherein convergence is determined by considering a maximum of the displacement and orientation as defined by
(DSₘₐₓ^{k}DVₘₐₓ^{k})=maxᵢ₌₁ⁿ(|Cᵢ^{k}−Pᵢ^{k}|,1−Tᵢ^{k}•Nᵢ^{k}),
where DSₘₐₓ^{k} is the maximum displacement and DVₘₐₓ^{k} is the maximum deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

28. The method of claim 23, wherein convergence is determined by considering an average of the displacement and orientation as defined by
$$( {DS}_{avg}^{k} , {DV}_{avg}^{k} ) - \frac{1}{N} {\sum }_{i = 1}^{n} ( \vert {C}_{i}^{k} - {P}_{i}^{k} \vert , 1 - {T}_{i}^{k} \cdot {N}_{i}^{k} )$$
where DS_{avg}^{k} is the average displacement and DV_{avg}^{k} is the average deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

29. The method of claim 23, further including calculating the lumen and wall contours on each cross-section, as well as other geometric information about these two contours.

30. The method of claim 23, further comprising the step of providing an endoluminal flight along the centerline of a vessel object, displaying hard plaque and soft plaque in different colors for differentiation from the vessel wall.

31. The method of claim 30, further comprising moving back and forth along the centerline by direct manipulation of a mechanism.

32. The method of claim 31, wherein the mechanism includes clicking or dragging a mouse along an overview of the entire vessel or scrolling a mouse wheel to scroll along the centerline of the vessel.

33. The method of claim 31, wherein the mechanism includes interactively tilting a viewpoint without leaving the centerline of the vessel.

34. A program storage device readable by a computer, tangibly embodying a program of instructions executable by the computer to perform the method steps for optimizing a vessel centerline in a digital image, said method comprising the steps of:
providing a digital image of a vessel wherein said image comprises a plurality of intensities corresponding to a domain of points in a D -dimensional space;
initializing a centerline comprising a plurality of points in the vessel;
determining a cross section of the vessel at each point in the centerline;
evaluating a center point for each cross section of the vessel; and
determining a refined centerline from the center points of each cross section.

35. The computer readable program storage device of claim 34, wherein the method steps of determining a cross section, evaluating a center point, and determining the refined centerline are repeated until the difference between each pair of successive refined centerlines is less than a predetermined quantity.

36. The computer readable program storage device of claim 34, wherein the cross section at a point in the centerline is determined by finding a cross section intersecting the centerline with a minimal area.

37. The computer readable program storage device of claim 36, wherein the cross section with minimal area is the cross section with the shortest lines intersecting the point in the centerline.

38. The computer readable program storage device of claim 34, wherein the cross section at a point on the centerline is perpendicular to a tangent vector of the centerline at the point on the centerline.

39. The computer readable program storage device of claim 38, the method further comprising the step of associating a reference frame to each cross section, wherein each said reference frame is defined by the centerline point in the cross section, and three orthogonal vectors that define an orientation of the reference frame, wherein the three orthogonal vectors include a tangent to the centerline at the centerline point, and two other orthogonal vectors in the plane of the cross section.

40. The computer readable program storage device of claim 39, wherein a first referenced frame can be determined from the centerline point in the cross section and the three orthogonal vectors, and a next reference frame can be determined by displacing the first reference frame to a next centerline point and rotating the displaced reference frame to align with the three orthogonal vectors of the cross section associated with the next centerline point.

41. The computer readable program storage device of claim 34, wherein evaluating a center point of each cross section comprises finding the contour of the cross section and using the contour to locate the centerpoint of the cross section.

42. The computer readable program storage device of claim 34, wherein evaluating a center point of each cross section comprises calculating a centroid of each cross section.

43. The computer readable program storage device of claim 42, wherein the method further comprises calculating the covariance matrix for each cross section, and calculating the eigenvalues and eigenvectors of the covariance matrix to determine the shape of the cross section.

44. The computer readable program storage device of claim 39, wherein determining a refined centerline further comprises the steps of:
connecting each successive pair of center points by a virtual spring whose force depends on the difference of the orientations of the pair of center points,
applying a stochastic perturbation to each virtual spring;
determining an optimized cross section of minimal area for each point on the centerline;
finding a center point of the optimized cross section; and
forming a refined centerline by connecting the center points of each optimized cross section.

45. The computer readable program storage device of claim 44, wherein the refined centerline is approximated by a least square cubic curve.

46. The computer readable program storage device of claim 44, wherein finding a center point of the optimized cross section comprises calculating a centroid of each optimized cross section.

47. The computer readable program storage device of claim 44, wherein the spring force connecting two successive centerpoint is defined by ƒ=k (1.0−T₀•T₁), wherein k is a constant and T₀ and T₁ are the tangent vectors of two successive center points.

48. The computer readable program storage device of claim 44, wherein the method further comprises the step of refining the centerline until it has converged to an optimal centerline, wherein convergence is determined from the displacement of each center point and the deviation of the orientation of each reference plane.

49. The computer readable program storage device of claim 48, wherein convergence is determined by considering a maximum of the displacement and orientation as defined by
(DSₘₐₓ^{k}DVₘₐₓ^{k})=maxᵢ₌₁ⁿ(|Cᵢ^{k}−Pᵢ^{k}|,1−Tᵢ^{k}•Nᵢ^{k}),
where DSₘₐₓ^{k} is the maximum displacement and DVₘₐₓ^{k} is the maximum deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

50. The computer readable program storage device of claim 48, wherein convergence is determined by considering an average of the displacement and orientation as defined by
$$( {DS}_{avg}^{k} , {DV}_{avg}^{k} ) = \frac{1}{N} {\sum }_{i = 1}^{n} ( \vert {C}_{i}^{k} - {P}_{i}^{k} \vert , 1 - {T}_{i}^{k} \cdot {N}_{i}^{k} )$$
where DS_{avg}^{k} is the average displacement and DV_{avg}^{k} is the average deviation of tangent vector at the k^{th} iteration, Cᵢ^{k} is the i^{th} updated center point, Pᵢ^{k} is the position of the i^{th} reference frame, Tᵢ^{k} is the i^{th} updated tangent direction and Nᵢ^{k} is the normal of the i^{th} reference frame at the k^{th} iteration.

51. The computer readable program storage device of claim 38, wherein the method further includes calculating the lumen and wall contours on each cross-section, as well as other geometric information about these two contours.

52. The computer readable program storage device of claim 34, wherein the method further comprises the step of providing an endoluminal flight along the centerline of a vessel object, displaying hard plaque and soft plaque in different colors for differentiation from the vessel wall.

53. The computer readable program storage device of claim 52, wherein the method further comprising moving back and forth along the centerline by direct manipulation of a mechanism.

54. The computer readable program storage device of claim 53, wherein the mechanism includes clicking or dragging a mouse along an overview of the entire vessel or scrolling a mouse wheel to scroll along the centerline of the vessel.

55. The computer readable program storage device of claim 53, wherein the mechanism includes interactively tilting a viewpoint without leaving the centerline of the vessel.
